(19) United States
(12) Patent Application Publication
(10) Pub. No.: US 2023/0390398 A1
(43) Pub. Date: Dec. 7, 2023

(54) METHODS AND COMPOSITIONS USEFUL FOR REDUCING BIOBURDEN IN WOUNDS

(71) Applicant: NEXT SCIENCE IP HOLDINGS PTY LTD, Chatswood, New South Wales (AU)

(72) Inventor: Matthew F. Myntti, St. Augustine, FL (US)

(73) Assignee: NEXT SCIENCE IP HOLDINGS PTY LTD, Chatswood, New South Wales (AU)

(21) Appl. No.: 18/031,644

(22) PCT Filed: Oct. 13, 2021
(86) PCT No.: PCT/US2021/054819

§ 371 Date: Apr. 21, 2022

Related U.S. Application Data
(60) Provisional application No. 63/091,421, filed on Oct. 14, 2020.

(51) Int. Cl. A61K 47/12; A61P 41/00; A61K 47/20

(52) U.S. Cl. CPC A61K 47/12; A61P 41/00; A61K 47/20

(57) ABSTRACT

A sterile, acidic liquid which has an effective solute concentration of from 0.3 to 0.6 Osm/L and a pH of from 3.7 to 4.2 can be used to treat the wound cavity of a mammalian subject. When used during surgeries performed on mammals, the liquid composition can be introduced to a surgery site wound cavity so as to reduce bioburden in that wound cavity. Advantageously, the composition need not be diluted or removed prior to approximation of the surgical wound.

### CROSS REFERENCE TO RELATED APPLICATIONS

This application claims priority to U.S. patent appl. No. 63/091,421 filed on Oct. 14, 2020, which is incorporated herein by reference.

### BACKGROUND INFORMATION

Microbes are found virtually everywhere, often in high concentrations, and are responsible for a significant amount of disease and infection.

Bacteria present special challenges because they can exist in a number of forms (e.g., planktonic, spore and biofilm) and their self-preservation mechanisms complicate or even confound efforts to treat and/or eradicate them. For example, bacteria in biofilms or spores are down-regulated (sessile) and not actively dividing, which makes them resistant to attack by those antibiotics and antimicrobials which attack bacteria during cell division.

In a biofilm, bacteria interact with and adhere to surfaces and form colonies which facilitate continued growth. The bacteria produce exopolysaccharide (EPS) and/or extracellular-polysaccharide (ECPS) macromolecules that keep them attached to the surface and form a protective barrier. Protection most likely can be attributed to the small diameters of the flow channels in the matrix, which restrict the size of molecules that can reach the underlying bacteria, and consumption of biocides through interactions with portions of the EPS/ECPS macromolecular matrix and bacterial secretions and waste products contained therein.

A number of recently described compositions have shown great efficacy against bacterial biofilms; see, for example, U.S. Pat. Nos. 8,940,792, 9,427,417, 10,477,860, etc. Nevertheless, preventing biofilm formation is preferable to treating one which has formed.

Any surface that is or becomes moist is subject to biofilm formation.

Animal tissue wounds present a good environment for growth of bacteria, and even biofilms. Extreme measures are taken to prevent biofilm formation in wounds because, once established, they are extremely difficult to eradicate in vivo and can cause life-altering, even lethal, infections. Additionally, any treatment of a bacterial infection, including a biofilm, must be gentle, thus complicating an already difficult problem.

In surgeries, wounds are intentionally created to permit access to areas below the dermis. Accordingly, surgical theaters are cleaned thoroughly and disinfected, surgical instruments are sterilized before use, the patient's dermis at the surgical site is disinfected, etc. Nevertheless, any bacteria which happen to be or become present in a surgical wound can establish a biofilm within about an hour.

Because of the remarkable ability of bacteria to so quickly establish themselves in biofilm form, surgical teams almost always lavage wound cavities prior to approximation of surgical site wounds.

Lavage compositions can be introduced manually, via gravity feed, via syringe, or under pressure, e.g., jet or pulsatile lavage. Normal saline solution remains the most commonly used lavage, although some surgeons use aqueous solutions of chlorhexidine gluconate (0.5% (w/v)), povidone iodine (typically at ˜0.35% (w/v)), or dilute hypochlorous acid; none of these has shown significant lethality toward biofilm-form microbes present in a surgical site wound cavity, however. All of the aforementioned lavage compositions other than normal saline must be removed from a surgical site wound cavity prior to approximation of the surgical site wound.

A lavage with excellent antimicrobial activity and lethality toward bacteria in a surgical site wound cavity now is available under the tradename Bactisure™ (Zimmer Biomet; Warsaw, Indiana). Unlike previous options, it has shown significant lethality toward bacteria in both planktonic and biofilm forms, but its composition, including inter alia extremely high osmolarity, mandates that it too be removed from a surgical site wound cavity prior to wound approximation.

Lavage removal is accomplished by flooding the surgical site wound cavity with normal saline (using any of the same techniques employed to introduce the lavage previously), thereby diluting the lavage. The diluted lavage often is evacuated via suction.

Dilution, typically followed by evacuation, adds time during which the surgical site wound remains open, lengthens the time which the surgical team must be in the surgical theater, and extends the period during which that surgical theater is unavailable for cleaning/disinfection and subsequent use, increasing cost while, in most instances, providing very little statistically significant improvement in outcomes.

An entirely new category of pre-approximation surgical site antimicrobial compositions, one which does not require dilution or rinsing away with normal saline but which is safe to mammalian tissue, is highly desirable. Such a composition preferably would be able to be left in place, at least in part and preferably altogether, so as to continue to act on bacteria that happen to be present in or that later arrive at the surgical site wound cavity.

### SUMMARY

Provided herein are compositions useful for treating wound cavities in mammalian subjects. The compositions are sterile, aqueous solutions which have effective solute concentrations of from 0.3 to 0.6 Osm/L and 3.7≤pH≤4.2.

The compositions can be used during surgeries performed on mammals. Prior to approximation of a surgical wound, an inventive composition introduced to the surgery site wound cavity can reduce bioburden therein. Advantageously, the composition need not be diluted or removed, in part or in whole, prior to the surgical wound being approximated.

In situations where a biofilm has formed or is in the process of forming in a surgical wound cavity, reduction in bioburden can involve (a) negatively impacting the integrity of the biofilm's protective EPS/ECPS macromolecules so that the entire structure can be dissolved, washed away, or otherwise prevented from becoming permanently ensconced in or on tissue located in the surgical wound cavity and/or (b) killing previously protected bacteria. In situations where a biofilm has yet to form in a surgical wound cavity, reduction in bioburden can involve killing planktonic bacteria. (Killing of bacteria typically does not occur through interruption or modification of a cellular process but, instead, via lysing of their cellular membranes as a result of osmotic pressure, membrane integrity disruption due to the presence of a surfactant, or some combination thereof.)

Other aspects of the invention will be apparent to the ordinarily skilled artisan from the detailed description that follows. To assist in understanding that description, certain definitions are provided immediately below, and these are intended to apply throughout unless the surrounding text explicitly indicates a contrary intention:
“comprising” means including, but not limited to, the listed ingredients or steps;
“consisting of” means including only the listed ingredients (or steps) and minor amounts of inactive additives or adjuvants;
“room temperature” means 20° to 25° C.;
“body temperature” means the average temperature of a mammal±1.5° C., for example, ˜35° to ˜38° C. for a North American human, ˜37° to ˜40° C. for a canine, etc.;
“polyacid” means a compound having at least two carboxyl groups and specifically includes dicarboxylic acids, tricarboxylic acids, etc.;
“pH” means the negative value of the base 10 logarithm of [H*] as determined by an acceptably reliable measurement method such as a properly calibrated pH meter, titration curve against a known standard, or the like;
“pKₐ;” means the negative value of the base 10 logarithm of a particular compound's acid dissociation constant;
“buffer” means a compound or mixture of compounds having an ability to maintain the pH of a solution to which it is added within relatively narrow limits;
“buffer precursor” means a compound that, when added to a mixture containing an acid, results in a buffer;
“electrolyte” means a compound that exhibits some dissociation when added to water;
“purified water” means water having a bacterial count and a level of endotoxins below those in tap water, well water, or spring water, either as-is or after a treatment such as softening or ion exchange;
“pharmaceutical grade” means a compound which meets a chemical purity standard established by a national or regional pharmacopeia;
“medication” means a substance which provides a therapeutic benefit to a treated subject;
“viscosifier” means a compound which decreases the speed at which a liquid spreads while still permitting that liquid some degree of flow at room temperature or higher;
“benzalkonium chloride” refers to any compound defined by the following general formula

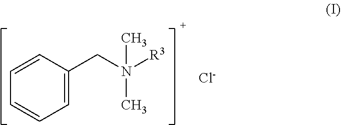

where R³ is a C₈-C₁₈ alkyl group, or any mixture of such compounds;
“effective solute concentration” is a measurement of the colligative property resulting from the number of moles of molecules (from nonelectrolyte) or ions (from electrolytes) present in a given volume solution, often presented in units of osmoles per liter;
“sterile,” when used in connection with a liquid composition and/or a container for such a liquid, means one which has been treated so as to kill any living organisms contained therein;
“substituted” means containing a heteroatom or functionality (e.g., hydrocarbyl group) that does not interfere with the intended purpose of the group in question;
“approximate,” when used in connection with a surgical procedure, means the closing of a surgical wound;
“wound cavity” means the area of a body which, although typically covered by the dermis, is capable of being contacted by a liquid introduced from an external source;
“microbe” means any type of microorganism including, but not limited to, bacteria, viruses, fungi, viroids, and prions;
“bioburden” means microbes and/or a substance produced, excreted or resulting from the presence of microbes;
“antimicrobial agent” means a substance having the ability to cause greater than a 90% (1 log) reduction in the number of one or more microbes;
“active antimicrobial agent” means an antimicrobial agent that is effective only or primarily during the active parts of the lifecycle, e.g., cell division, of a microbe;
“dwell time” means the amount of time that a composition is allowed to contact a surface and/or a microbe on such a surface; and
“healthcare” means involved in or connected with the maintenance or restoration of the health of the body or mind.

Throughout this document, unless the surrounding text explicitly indicates a contrary intention, all values given in the form of percentages are w/v, i.e., grams of solute per liter of composition and pH values are those which can be obtained from any of a variety of potentiometric techniques employing a properly calibrated electrode.

The relevant portion(s) of any specifically referenced patent and/or published patent application are incorporated herein by reference.

### DETAILED DESCRIPTION OF ILLUSTRATIVE EMBODIMENTS

The composition is described first in terms of its properties and components, many of which are widely available and relatively inexpensive, and then in terms of certain uses.

The composition includes solvent and solute components.

The solvent component is primarily water, typically purified water. (Instances where water other than purified water might be employed are discussed below.) Relative to the total volume of the solvent component, purified water constitutes at least 95%, often at least 97%, and typically at least 99% (all w/v) thereof. On a per liter basis, a composition includes from ˜925 to ˜975 mL, commonly from ˜937 to ˜972 mL, more commonly from ˜950 to ˜970 mL, and typically 960±5 mL purified water.

Although not preferred, the solvent component can include small volumes of one or more organic liquids listed on the U.S. Food and Drug Administration inactive ingredients list, non-limiting examples of which include ethanol and propylene glycol. Where more than one organic liquid is included, the liquids should be unreactive toward one another. The organic liquids can constitute no more than 5%, preferably no more than 3%, and most preferably no more than 1% (all w/v) of the solvent component.

A preferred solvent component is 100% purified water.

Each sub-component of the solute component preferably is provided in pharmaceutical grade form, particularly where the composition is to be used in a surgical theater.

The composition is acidic, which means that at least one of the sub-components of the solute component must be an acid. Preferred acids are those which have relatively high pKₐ values, i.e., are not considered to be particularly strong acids.

Examples of potentially useful weak acids include monoprotic acids such as formic acid, acetic acid and substituted variants (e.g., hydroxyacetic acid, chloroacetic acid, dichloroacetic acid, phenylacetic acid, and the like), propanoic acid and substituted variants (e.g., lactic acid, pyruvic acid, and the like), any of a variety of benzoic acids (e.g., mandelic acid, chloromandelic acid, salicylic acid, and the like), glucuronic acid, and the like; diprotic acids such as oxalic acid and substituted variants (e.g., oxamic acid), butanedioic acid and substituted variants (e.g., malic acid, aspartic acid, tartaric acid, citramalic acid, and the like), pentanedioic acid and substituted variants (e.g., glutamic acid, 2-ketoglutaric acid, and the like), hexanedioic acid and substituted variants (e.g., mucic acid), butenedioic acid (both cis and trans isomers), iminodiacetic acid, phthalic acid, and the like; triprotic acids such as citric acid, 2-methylpropane-1,2,3-tricarboxylic acid, benzenetricarboxylic acid, nitrilotriacetic acid, and the like; tetraprotic acids such as prehnitic acid, pyromellitic acid, and the like; and even higher degree acids (e.g., penta-, hexa-, heptaprotic, etc.). Where a tri-, tetra-, or higher acid is used, one or more of the carboxyl protons can be replaced by cationic atoms or groups (e.g., alkali metal ions), which can be the same or different.

Citric acid constitutes a preferred acid because mammalian bodies have such familiarity with and tolerance toward it due to its use and regeneration as part of the Krebs cycle. Those solute components which include citric acid, particularly those which have citric acid as their sole acid, are preferred.

The amount of any given acid employed can be determined from the target pH of a given composition and the pKₐ value(s) of the chosen acids in view of the type and amounts of compound(s), if any, utilized to achieve the desired effective solute concentration in the composition.

Both to ensure that the pH of the composition is not too low and also to increase its effective solute concentration, the solute component also includes a conjugate base of at least one of the foregoing weak acids. Although not required, use of conjugate base(s) of the particular acid(s) employed is preferable.

Upon dissociation, conjugate bases, e.g., salt(s) of one or more of the acid(s), increase the effective amount of solutes in the composition without greatly impacting the molar concentration of hydronium ions while, simultaneously, act to buffer the pH of the composition. The identity of the countercation portion of the salt(s) is not believed to be particularly critical, with common examples including ammonium ions and alkali metals, with the latter being preferred countercations.

Where a conjugate base of polyacid is used, all or fewer than all of the H atoms of the carboxyl groups can be replaced with cationic atoms or groups, which can be the same or different. For example, mono-, di- and trisodium citrate all constitute potentially useful buffer precursors, whether used in conjunction with citric acid or another organic acid. However, because trisodium citrate has three available basic sites, it has a theoretical buffering capacity up to 50% greater than that of disodium citrate (which has two such sites) and up to 200% greater than that of sodium citrate (which has only one such site).

Like the acid(s) described above, the amount of conjugate base(s) can be determined based on the desired composition pH and effective solute concentration.

Many organic acids and their conjugate bases can be provided in either anhydrous or hydrate forms. The particular form of these materials does not impact utility or efficacy. Because all solutes are added to a solvent component that is all or almost all purified water, any water of hydration in the solute(s) merely becomes part of the solvent component.

The amounts of acid(s) and conjugate base(s) included in the solute component are added at levels that provide two important compositional characteristics, neither one of which is dependent on the particular materials which provide them.

The first such characteristic is pH. The present composition has a pH of from 3.7 to 4.2. A composition which has an even lower pH is quite likely to be even more effective in terms of disrupting EPS/ECPS macromolecules and in killing bacteria; however, this increased efficacy comes at a cost of decreased biocompatibility. Conversely, a composition having a pH>4.2 would have even greater biocompatibility, albeit at the cost of lower efficacy.

Within the permitted pH range, a pH of from 3.85 to 4.05 is preferred, with pH=3.95±0.1 or even 0.05 being particularly preferred.

The second important compositional characteristic is effective solute concentration, which induces sufficient osmotic pressure across a bacterium's cortical membrane to lead to lysis. This ability to induce osmotic pressure is independent of the particular identity or nature of individual compounds of the solute component, although smaller molecules are generally more effective than larger molecules due to solvent capacity (i.e., the ability to (typically) include more small molecules in a given amount of solvent component than an equimolar amount of larger molecules) and ease of transport across cortical membranes.

The present composition has an effective solute concentration of from ˜300 to ˜700 mOsm/L. A composition which has an effective solute concentration greater than ˜700 mOsm/L could be even more effective in terms of lethality toward bacteria; however, this increased efficacy comes at a cost of decreased biocompatibility, specifically, tissue inflammation.

The upper limit of the effective solute concentration can be impacted by the area of the body in which it is intended for use. For example, some studies have indicated that compositions having effective solute concentrations above ˜600 mOsm/L can cause unacceptable results (e.g., irritation and swelling) in the human peritoneal cavity. However, other studies have indicated that compositions having effective solute concentrations of ˜600 mOsm/L are better tolerated than lower concentration solutions when used in and around a joint, e.g., a shoulder.

Within the aforementioned permitted effective solute concentration range, preferred ranges are (1) for peritoneal usage, from ˜350 to ˜590 mOsm/L, particularly ˜400 to ˜580 and ˜450 to ˜575 mOsm/L, and (2) for non-peritoneal usage, from ˜450 to ˜680 mOsm/L, particularly ˜460 to ˜650 and ˜470 to ˜635 mOsm/L. A preferred overall range is from 450 to 675 mOsm/L.

Effective solute concentration can be calculated at a given compositional pH, with some free calculation tools being available online. No such calculation is absolute due to an increasing potential for reassociation of previously dissociated solutes as effective solute concentration increases. Nevertheless, the effective solute concentration values and ranges set forth above are theoretical maxima based on full dissociation.

Because it is a colligative property, effective solute concentration can be determined by techniques such as vapor pressure lowering, boiling point elevation, freezing point depression, and membrane osmometry. Because of their impact on properties such as boiling point and freezing point, where a particular composition happens to include one or more organic liquids, a tested composition which includes an equivalent volume of purified water in place of the organic liquid(s) should be used when performing one of the foregoing techniques so as to determine effective solute concentration.

Using citric acid and a citrate that includes three alkali metal ions as an exemplary acid and conjugate base pair, acceptable values for the aforedescribed compositional characteristics can be achieved (or at least approached, to permit achievement via the type of minor modification described below) using from 25 to 40 g/L citric acid and from 30 to 45 g/L of a citrate. Where anhydrous citric acid and trisodium citrate dihydrate are utilized, a preferred embodiment (for peritoneal usage) can be provided from 30 to 35 g/L of the acid and 34 to 38 g/L of the citrate, while another preferred embodiment (for joint usage) can be provided from 33 to 38 g/L of the acid and 37 to 42 g/L of the citrate.

Importantly, the particular species of acid and citrate discussed in the preceding paragraph need not be utilized. An ordinarily skilled artisan desiring to use a hydrate version of the acid, an anhydrous version of the citrate, a citrate having fewer than three alkali metal atoms (i.e., mono- or disodium citrate) readily can calculate amounts of each that will provide compositions having acceptable values for the aforedescribed compositional characteristics.

The solute component also includes one or more surface active agents that bear some type of ionic charge. Of these, anionic and cationic surfactants are preferred over zwitterionic surfactants. A composition should not include surfactant types that are incompatible, i.e., anionic with cationic or zwitterionic with either anionic or cationic.

Smaller molecules generally are preferred over larger sized surfactants. The size of side-groups attached to the polar head can influence the efficacy of ionic surfactants, with larger sized groups and more side groups on the polar head potentially decreasing its efficacy.

Potentially useful anionic surfactants include, but are not limited to, ammonium lauryl sulfate, dioctyl sodium sulfosuccinate, perfluorobutanesulfonic acid, perfluorononanoic acid, perfluorooctanesulfonic acid, perfluorooctanoic acid, potassium laurylsulfate, sodium dodecylbenzenesulfonate, sodium laureth sulfate, sodium lauroyl sarcosinate, sodium myreth sulfate, sodium pareth sulfate, sodium stearate, sodium chenodeoxycholate, N-lauroylsarcosine sodium salt, lithium dodecyl sulfate, 1-octanesulfonic acid sodium salt, sodium cholate hydrate, sodium deoxycholate, sodium dodecyl sulfate (SDS, also called sodium lauryl sulfate (SLS)), sodium glycodeoxycholate, and the alkyl phosphates set forth in U.S. Pat. No. 6,610,314. (Although sodium is used as the countercation in most of the foregoing exemplary anionic surfactants, other alkali metal ions can be used in its place.) SDS is a particularly preferred option.

Potentially useful cationic surfactants include, but are not limited to, cetylpyridinium chloride (CPC), cetyl trimethylammonium chloride, benzethonium chloride, 5-bromo-5-nitro-1,3-dioxane, dimethyldioctadecylammonium chloride, cetrimonium bromide, dioctadecyldimethylammonium bromide, tetradecyltrimethyl ammonium bromide, benzalkonium chloride (BZK), hexadecylpyridinium chloride monohydrate and hexadecyltrimethylammonium bromide.

Potentially useful zwitterionic surfactants include sulfonates (e.g. 3-[(3-cholamidopropyl)dimethylammonio]-1-propanesulfonate), sultaines (e.g. cocamidopropyl hydroxysultaine), betaines (e.g. cocamidopropyl betaine), and phosphates (e.g. lecithin).

Although not preferred, nonionic surfactant(s) can be included.

For other potentially useful surface active materials, the interested reader is directed to any of a variety of other sources including, for example, U.S. Pat. Nos. 4,107,328, 6,953,772, 7,959,943, and 8,940,792.

The amount(s) of surfactant(s) included is limited to some extent by the target effective solute concentration and compatibility with other subcomponents of the solute component. The total amount of surfactant present in the composition can range from ˜0.07 to ˜0.19% (w/v), typically ˜0.075 to ˜0.15% (w/v), preferably 1±0.25 g/L or 0.95±0.2 g/L.

If the acid(s), conjugate base(s) and surfactant(s) do not provide a desired effective solute concentration, one or more electrolytes, particularly ionic compounds (salts), can be added; see, e.g., U.S. Pat. No. 7,090,882, for a list of potentially useful electrolytes.

Not preferred but permissible in the solute component is one or more inactive ingredients (additives) approved by the U.S. Food & Drug Administration, available as a zipped text file at https://www.fda.gov/media/72482/download (link active as of filing date of this application).

A typical manner of making a composition involves adding the solute sub-components, either separately or as an admixture, to the solvent component (or to the water sub-component of the solvent component, followed by addition of the organic liquid(s)). This addition can be done with the benefit of one or both of stirring and heating of the mixing container.

If assurance of a targeted pH range is considered important, once the solute component has been added to the solvent component, very small aliquots of a concentrated acid (e.g., 1M HCl) or concentrated base (e.g., 1M KOH) can be used to lower or raise the composition's pH into the targeted range.

The following table provides an ingredient list for providing exemplary compositions according to the present invention, with amounts being given in grams.

TABLE 1
Formulations for exemplary compositions

| | | Amount, | Amount, |
|---|---|---|---|
| | Preferred species | generally | preferred |
| organic acid | citric acid | 30-35 | 31-34 |
| conjugate base of | citrate (e.g., trisodium | 30-40 | 34-38 |
| organic acid | citrate) | | |
| ionic surfactant | SDS | 0.85-1.15 | 0.9-2.1 |
| water | USP water | 950-975 | 955-970 |

Various embodiments of the present invention have been provided by way of example and not limitation. As evident from the foregoing tables, general preferences regarding features, ranges, numerical limitations and embodiments are, to the extent feasible and as long as not interfering or incompatible, envisioned as being capable of being combined with other such generally preferred features, ranges, numerical limitations and embodiments.

Given that reducing bioburden in surgical site wound cavities is a contemplated usage of the composition, it typically will be provided to a surgical theater packaged in sterile form, i.e., its container having been subjected to sufficient heat, radiation, etc., so as to render the composition sterile (aseptic).

Typical containers include bags and bottles of a type similar to those used to deliver liquids such as saline solutions in surgical theaters.

The container has one or more access points, for example, a port covered and protected by a septum. Where a container has more than one such access point, one of the access points can be used to introduce at least one medication to the interior of the container prior to the container's contents, i.e., the composition and medication(s), being evacuated from the container through another of the access points. Introduction of medication into the interior of the container can be accomplished by syringe injection through a septum.

Non-limiting categories of medications which can be added to the composition include
steroids such as hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, and desonide;
antibiotics such as Amikacin, Amoxicillin, Ampicillin, Arsphenamine, Azithromycin, Azlocillin, Aztreonam, Bacitracin, Capreomycin, Cefaclor, Cefadroxil, Cefalexin, Cefamandole, Cefazolin, Cefdinir, Cefditoren, Cefepime, Cefixime, Cefmetazole, Cefonicid, Cefoperazone, Cefotaxime, Cefotetan, Cefoxitin, Cefpodoxime, Cefprozil, Ceftaroline, Ceftazidime, Ceftibuten, Ceftizoxime, Ceftobiprole, Ceftriaxone, Cefuroxime, Cephalosporins, Cephalothin, Cephapirin, Cephradine, Chloramphenicol, Ciprofloxacin, Clarithromycin, Clindamycin, Clofazimine, Colistin, Cycloserine, Dalbavancin, Dapsone, Daptomycin, Dicloxacillin, Doripenem, Doxycycline, Enoxacin, Ertapenem, Erythromycin, Ethambutol, Ethionamide, Fidaxomicin, Flucloxacillin, Fosfomycin, Furazolidone, Fusidic acid, gatifloxacin, Geldanamycin, gemifloxacin, Gentamicin, grepafloxacin, Halicin, Herbimycin, Imipenem/Cilastatin, Isoniazid, Kanamycin, levofloxacin, Lincomycin, Linezolid, lomefloxacin, Loracarbef, Mafenide, Malacidins, Meropenem, Metacycline, methicillin, Metronidazole, Mezlocillin, Minocycline, Moxalactam, moxifloxacin, Mupirocin, nadifloxacin, Nafcillin, Nalidixic acid, Neomycin, Netilmicin, Nitrofurantoin, norfloxacin, ofloxacin, Omadacycline, Oritavancin, Oxacillin, Oxazolidinones, Oxytetracycline, Paromomycin, Penicillin G, Penicillin V, Piperacillin, Piperacillin/tazobactam, Platensimycin, Polymyxin B, polypeptides, Posizolid, pyrazinamide, Quinupristin/Dalfopristin, Radezolid, Rifabutin, Rifampicin, Rifapentine, Rifaximin, Roxithromycin, Serotonin Syndrome, silver sulfadiazine, sparfloxacin, Spectinomycin, Spiramycin, Streptomycin, Sulfacetamide, Sulfadiazine, Sulfadimethoxine, Sulfamethizole, Sulfamethoxazole, Sulfanilimide, Sulfasalazine, Sulfisoxazole, Sulfonamidochrysoidine, Tedizolid, Teicoplanin, Teixobactin, Telavancin, Telithromycin, temafloxacin, Temocillin, tetracycline, Thiamphenicol, Thrombocytopenia, Ticarcillin, Ticarcillin/clavulanate, Tigecycline, Tinidazole, Tobramycin, Torezolid, Trimethoprim, Trimethoprim/sulfamethoxazole, trovafloxacin, and Vancomycin;
anticoagulants such as heparin, Apixaban, Dabigatran, Edoxaban, Enoxaparin, Rivaroxaban, and warfarin;
clotting promoters such as aprotinin, epsilon-aminocaproic acid, aminomethylbenzoic acid, and tranexamic acid;
antifungals such as Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, miconazole, Omoconazole, Oxiconazole, Sertaconazole, Sulconazole, Tioconazole, Albaconazole, Efinaconazole, Epoxiconazole, fluconazole, Isavuconazole, Itraconazole, Posaconazole, Propiconazole, Ravuconazole, Terconazole, Voriconazole, Abafungin, amorolfin, butenafine, naftifine, terbinafine, Anidulafungin, Caspofungin, Micafungin, Aurones, benzoic acid, Ciclopirox, Flucytosine or 5-fluorocytosine, Griseofulvin, Haloprogin, Tolnaftate, undecylenic acid, Triacetin, Crystal violet, Orotomide, Miltefosine, potassium iodide, Nikkomycin, copper(II) sulfate, selenium disulfide, sodium thiosulfate, Piroctone olamine, Iodoquinol (diiodohydroxyquin), Acrisorcin, zinc pyrithione, and sulfur;
anesthetics such as lidocaine, benzocaine, butamben, dibucaine, oxybuprocaine, pramoxine, proxymetacaine and tetracaine; and
analgesics such as 2-(4-(2-methylpropyl)phenyl)propanoic acid (i.e., ibuprofen), capsaicin, diclofenac, lidocaine, methyl salicylate, and trolamine.

Such medications preferably are delivered in purified water. Because some of the aforementioned classes of medications, or certain species within a given class, can have limited solubility in water, delivery in an organic liquid (or a solution which includes an organic liquid) might be necessary or desirable. In such cases, the considerations regarding type and amount of such organic liquid(s) set forth above should be taken into account.

When adding one or more of such medications to the composition container, the solubility limits of the medications at the composition's temperature necessarily must be taken into account.

Prior to evacuation of the container contents, the container and those contents can be warmed. While such warming can assist in assuring that all solute components are fully dissolved, it also provides the side benefit of bringing the temperature of the composition closer to that of the surgical patient's internal temperature. In view of the latter, the temperature of the composition preferably is within 5° C. of the body temperature of the particular type of mammal on which the surgery is being performed. (In extremely hot climates, bringing the temperature of the composition to within the desired range might require cooling rather than warming.)

Where a medication is to be introduced into the container prior to the container contents being evacuated, the aforementioned temperature adjustment can occur before or after introduction of the medication to the composition.

Transferring the composition from the interior of the container to the surgical wound cavity of the patient can be accomplished in numerous ways.

One option involves decanting the contents of the container into a sterile basin by means of a tube with a spiked end. Evacuation of container contents typically occurs solely through the force of gravity. Once decanted, a medical professional, e.g., surgeon, can pour the decanted composition from the basin over and into the wound cavity.

A variation of the foregoing involves use of a bulb syringe (or similar) by the medical professional to better direct flow of the composition into and around the wound cavity.

Where the container is a bottle (typically packaged in a thermoformed polymeric tray with a removable, polymeric lid), its contents can be evacuated similarly to the option just described. If the bottle is sealed, the seal is removed and a cap with nozzle applied. (If the bottle includes an integrated nozzle, this step can be avoided.) The medical professional can use the nozzle to direct composition flow into and around the wound cavity similarly to the manner employed with a bulb syringe.

Another option involves use of a device that can deliver the composition under pressure, e.g., a pulsed or jet lavage delivery system such as Interpulse™ pulsed lavage system (Stryker; Kalamazoo, Michigan) or Pulsavac™ Plus lavage system (Zimmer Biomet; Warsaw, Indiana). Similar to the gravity feed option described above, the composition can be accessed using a tube with a spiked end, with the other end of that tube being attached to and feeding the delivery instrument. A medical professional using the wand or gun portion of the delivery instrument directs flow of the composition into and rinses the surgical wound cavity.

Regardless of how introduced, the amount of composition delivered into the surgical wound cavity can vary from as little as a few milliliters for small surgical sites up to 0.5, 1, 1.5 or 2 L (optionally delivered in more than one aliquot).

Although the composition is designed for use prior to a surgical wound being approximated at the end of a surgery, this is not limiting. The composition can be used at any point during a surgical procedure, for example, in the washing away of debris of one step prior to moving on to the next step of the procedure.

Regardless of when used during a surgical procedure, the composition does not require rinsing or suctioning; some or all can remain in the surgical wound cavity during and after surgical wound approximation.

At least a portion of the introduced composition remains behind after approximation of the surgical wound. The amount of composition remaining in the former surgical wound cavity can be as little as little as necessary to provide a coating on exposed (internal) tissues (0.5 to 10 mL) to as much composition as was introduced during the surgery. The fact that some composition remains behind means that it can work to reduce bioburden during the process of approximation and until such portion is biosorbed.

In situations where a not insubstantial amount of composition is introduced to the surgical wound cavity during the surgical procedure (e.g., ˜100 mL or more), partial removal via suction can be preferred. In situations where a substantial amount of composition is introduced to the surgical wound cavity during the surgical procedure (e.g., ˜250 mL or more), partial removal via suction is preferred. (Some composition might exit the surgical wound cavity via normal outflow during or after introduction.) Many pressure delivery (e.g., pulsed lavage) systems have integrated suctioning, i.e., the same device that introduces the composition is designed to also remove it with suction.

Importantly, removal by suction need not be preceded and/or followed by a saline solution rinse, i.e., the composition is sufficiently gentle and biocompatible that its continued presence in a wound cavity does not result in significant deleterious effects.

The amount of composition that remains in the surgical wound cavity during and after wound approximation typically ranges from a few milliliters up to ˜250 mL, with the amount largely depending on whether partial removal via suction has been employed.

When wounds are closed, edges of the wound are approximated by standard techniques including sutures, staples, adhesive(s) and the like. Approximation can be complete or partial, e.g., incorporation of a wound drain.

After a surgical wound is approximated, it and the surrounding area can be rinsed with a disinfecting solution and/or covered with a sterile protecting layer (optionally with an antimicrobial gel or cream such as BLASTX™ wound gel or SURGX™ sterilized gel, both available from Next Science (Jacksonville, Florida)).

The foregoing has focused on a common usage of the inventive compositions, i.e., use in surgical theaters. The compositions have additional utilities and methods of use, however, including specifically emergency medical care for open wounds, regardless of whether in hospital emergency departments, during patient transport (e.g., ambulance, life flight, etc.), or on the battlefield. In each of these situations, wound closure soon after introduction of the composition to a wound cavity is unlikely; instead, the composition can be introduced as soon as possible to the wound cavity, where it will remain for bioburden reduction purposes until more thorough wound treatment can be undertaken.

In situations such as emergency departments and patient transport, the composition often will be packaged similarly to that described above with respect to surgical theater usage. This might also be true for battlefield usage, but not necessarily so. For example, a medic or corpsman might prefer to carry the subcomponents of the solute component of the composition in a packet, sachet, or other container, then add them to an appropriate amount of water (which need not, and often will not, be purified) or vice versa. Once constituted, the composition then can be introduced directly into a wound cavity. Additional composition can be added during patient transport.

As discussed above, compositions described herein advantageously reduce bioburden in wound cavities.

The reduction in bioburden can be quantified, for example by assaying a change in bacterial colony forming units (CFU) before and after treatment with the composition. When bacteria are killed upon exposure to the composition, the change in CFU reflects the change in the number of living bacteria.

Alternatively or additionally, a biofilm might lose integrity due to exposure of the protective EPS/ECPS to the composition, such that the some or all of the structure can be dissolved, washed away, or otherwise prevented from becoming permanently ensconced in or on tissue located in the surgical wound cavity. In such a situation, the change in CFU reflects the loss of such bacteria (even though not killed by the composition) due to the dissolving, washing away, or otherwise being prevented from becoming permanently ensconced.

When quantified by a change in CFU, the reduction in bioburden may be a reduction of at least 90% (1 log) in CFU, preferably a reduction of at least 99% (2 log) in CFU, more preferably a reduction of at least 99.9% (3 log) in CFU, or even more preferably a reduction of at least 99.99% (4 log) in CFU. This reduction in bioburden typically is measured over a time representative of the method for treating the wound. For example, the change in CFU may be measured starting from the time the composition is introduced to the wound cavity until the time the wound is approximated. Alternatively, the time may be specified as a specific value, such as 60 seconds, 120 seconds, 240 seconds, or 300 seconds.

The following embodiments are specifically contemplated. An embodiment relating to a method of use involving a composition is intended to be read as also relating to the composition for use in that method.

Embodiment [1] relates to a method for treating a wound cavity in a mammalian subject, the method comprising:
a) providing a sterile, acidic liquid composition, the composition consisting of solvent and solute components and having an effective solute concentration of from 0.3 to 0.7 Osm/L and a pH of from 3.7 to 4.2;
b) prior to approximation of the wound, introducing the composition to the wound cavity; and
c) permitting at least a portion of the composition to reduce bioburden in the wound cavity during and after approximation of the wound.

Embodiment [2] relates to the method of Embodiment [1] wherein the composition has an effective solute concentration of from 350 to 590 mOsm/L.

Embodiment [3] relates to the method of Embodiment [1] wherein the composition has an effective solute concentration of from 450 to 680 mOsm/L.

Embodiment [4] relates to any one of the methods of Embodiments [1] to [3] wherein the composition has a pH of from 3.85 to 4.05.

Embodiment [5] relates to the any one of the methods of Embodiments [1] to [4] wherein the composition is undiluted prior to the wound approximation.

Embodiment [6] relates to any one of the methods of Embodiments [1] to [4] wherein a portion of the composition is removed or diluted prior to the wound approximation.

Embodiment [7] relates to any one of the methods of Embodiments [1] to [6] wherein the solvent component consists of purified water.

Embodiment [8] relates to any one of the methods of Embodiments [1] to [7] wherein all solutes in the solute component are pharmaceutical grade.

Embodiment [9] relates to any one of the methods of Embodiments [1] to [7] wherein the solute component comprises a buffer system and an ionic surfactant.

Embodiment [10] relates to any one of the methods of Embodiments [1] to [7] wherein the solute component consists of a buffer system and an ionic surfactant.

Embodiment [11] relates to any one of the methods of Embodiments [9] to [10] wherein the buffer system comprises dissociation products of a carboxylic acid and a conjugate base of a carboxylic acid.

Embodiment [12] relates to any one of the methods of Embodiments [9] to [10] wherein the buffer system consists of dissociation products of at least one carboxylic acid and at least one conjugate base of at least one carboxylic acid.

Embodiment [13] relates to any one of the methods of Embodiments [11] to [12] wherein the carboxylic acid is citric acid and wherein the conjugate base is a citrate.

Embodiment [14] relates to the method of Embodiment [13] wherein the buffer system comprises dissociation products of from 25 to 40 g/L citric acid and from 30 to 45 g/L of a citrate that comprises three alkali metal ions.

Embodiment [15] relates to any one of the methods of Embodiments [9] to [10] wherein the composition comprises from 0.7 to 1.9 g/L ionic surfactant.

Embodiment [16] relates to any one of the methods of Embodiments [9] to [10] and [15] wherein the ionic surfactant is an anionic surfactant.

Embodiment [17] relates to any one of the methods of Embodiments [1] to [16] wherein the providing step involves delivery of the composition in a container that comprises at least one access point.

Embodiment [18] relates to the method of Embodiment [17] wherein the container comprises multiple access points, the method further comprising adding at least one medication to the composition prior to the introducing step.

Embodiment [19] relates to any one of the methods of Embodiments [1] to [18] further comprising, prior to the introducing step, adjusting the temperature of the composition to within 5° C. of body temperature.

Embodiment [20] relates to any one of the methods of Embodiments [1] to [19] wherein the composition is introduced by an emergency medical service provider, wherein at least some of the bioburden reduction occurs prior to or during transport of the mammalian subject.

Embodiment [21] relates to any one of the methods of Embodiments [1] to [19] wherein the composition is introduced during an operation in a surgical theater, wherein the bioburden reduction occurs before, during and after the wound approximation.

Embodiment [22] relates to a process for treating a wound cavity in a mammalian subject, the process comprising:
a) providing a container that comprises at least one access point that holds a sterile, acidic liquid composition, the composition having an effective solute concentration of from 0.3 to 0.7 Osm/L and a pH of from 3.7 to 4.2, the composition consisting of
   1) a solvent component consisting of purified water, and
   2) a solute component comprising a buffer system and from 0.7 to 1.9 g/L ionic surfactant,
b) prior to approximation of the wound, introducing the composition to the wound cavity; and
c) permitting at least a portion of the composition to reduce bioburden in the wound cavity during and after approximation of the wound.

Embodiment [23] relates to the process of Embodiment [22] wherein the composition has an effective solute concentration of from 350 to 590 mOsm/L.

Embodiment [24] relates to the process of Embodiment [22] wherein the composition has an effective solute concentration of from 450 to 680 mOsm/L.

Embodiment [25] relates to any one of the processes of Embodiments [22] to [24] wherein the composition has a pH of from 3.85 to 4.05.

Embodiment [26] relates to any one of the processes of Embodiments [22] to [25] wherein the composition is undiluted prior to the wound approximation.

Embodiment [27] relates to any one of the processes of Embodiments [22] to [25] wherein a portion of the composition is removed or diluted prior to the wound approximation.

Embodiment [28] relates to any one of the processes of Embodiments [22] to [27] wherein all solutes in the solute component are pharmaceutical grade.

Embodiment [29] relates to any one of the processes of Embodiments [22] to [28] wherein the solute component consists of a buffer system and an ionic surfactant.

Embodiment [30] relates to any one of the processes of Embodiments [22] to [29] wherein the buffer system comprises dissociation products of a carboxylic acid and a conjugate base of a carboxylic acid.

Embodiment [31] relates to any one of the processes of Embodiments [22] to [29] wherein the buffer system consists of dissociation products of at least one carboxylic acid and at least one conjugate base of at least one carboxylic acid.

Embodiment [32] relates to any one of the processes of Embodiments [30] to [31] wherein the carboxylic acid is citric acid and wherein the conjugate base is a citrate.

Embodiment [33] relates to the process of Embodiment [32] wherein the buffer system comprises dissociation products of from 25 to 40 g/L citric acid and from 30 to 45 g/L of a citrate that comprises three alkali metal ions.

Embodiment [34] relates to any one of the processes of Embodiments [22] to [33] wherein the ionic surfactant is an anionic surfactant.

Embodiment [35] relates to the process of Embodiment [34] wherein the anionic surfactant is sodium lauryl sulfate.

Embodiment [36] relates to any one of the processes of Embodiments [22] to [35] wherein the container comprises multiple access points, the process further comprising adding at least one medication to the composition prior to the introducing step.

Embodiment [37] relates to any one of the processes of Embodiments [22] to [36] further comprising, prior to the introducing step, adjusting the temperature of the composition to within 5° C. of body temperature.

Embodiment [38] relates to any one of the processes of Embodiments [22] to [37] wherein the composition is introduced by an emergency medical service provider, wherein at least some of the bioburden reduction occurs prior to or during transport of the mammalian subject.

Embodiment [39] relates to any one of the processes of Embodiments [22] to [37] wherein the composition is introduced during an operation in a surgical theater, wherein the bioburden reduction occurs before, during and after the wound approximation.

Embodiment [40] relates to the process of Embodiment [39] wherein the mammalian subject is human.

Embodiment [41] relates to a method for treating a surgical site in a mammalian subject, the method consisting of:
a) providing a container that comprises at least one access point that holds a sterile, acidic liquid composition, the composition having an effective solute concentration of from 450 to 675 mOsm/L and a pH of from 3.85 to 4.05, the composition consisting of
   1) a solvent component consisting of purified water, and
   2) a solute component that consists of
      (A) a buffer system that comprises dissociation products of citric acid and at least one citrate,
      (B) from 0.75 to 1.25 g/L anionic surfactant, and
      (C) optionally, one or more adjuvants selected from dyes, preservatives and viscosifiers;
   b) where the container comprises more than one access point, optionally adding at least one medication to the composition;
   c) prior to approximation of the surgical site opening, introducing the composition to the opening; and
   d) permitting at least a portion of the composition to reduce bioburden at the surgical site during and after approximation.

Embodiment [42] relates to the method of Embodiment [41] wherein the anionic surfactant is sodium lauryl sulfate.

Embodiment [43] relates to any one of the methods of Embodiments [41] to [42] wherein the buffer system comprises dissociation products of from 30 to 38 g/L citric acid and of from 34 to 42 g/L of trisodium citrate.

Embodiment [44] relates to any one of the methods of Embodiments [41] to [43] wherein the container is provided at a temperature that is within 5° C. of the body temperature of the mammalian subject.

Embodiment [45] relates to any one of the methods of Embodiments [41] to [44] wherein the composition is undiluted prior to the wound approximation.

Embodiment [46] relates to any one of the methods of Embodiments [41] to [44] wherein a portion of the composition is removed or diluted prior to the wound approximation.

Embodiment [47] relates to a sterile, acidic liquid composition useful to reduce bioburden in a surgical site of a mammalian subject, the composition being provided in a container that comprises at least one access point, the composition consisting of
a) a solvent component consisting of purified water, and
b) a solute component that consists of
   1) a buffer system that comprises dissociation products of
      (A) from 30 to 38 g/L citric acid and
      (B) from 34 to 42 g/L of at least one citrate,
   2) from 0.75 to 1.25 g/L anionic surfactant, and
   3) optionally, one or more adjuvants selected from dyes, preservatives and viscosifiers.

Embodiment [48] relates to the composition of Embodiment [47] wherein the buffer system consists of dissociation products of the citric acid and the at least one citrate.

Embodiment [49] relates to the composition of Embodiment [48] wherein the at least one citrate comprises trisodium citrate.

Embodiment [50] relates to the composition of Embodiment [48] wherein the at least one citrate consists of trisodium citrate.

Embodiment [51] relates to the composition of any one of Embodiments [47] to [50] wherein the anionic surfactant is sodium lauryl sulfate.

Embodiment [52] relates to the composition of any one of Embodiments [47] to [51] having an effective solute concentration of 525±50 mOsm/L.

Embodiment [53] relates to the composition of Embodiment [52] wherein the effective solute concentration is 525±25 mOsm/L.

Embodiment [54] relates to the composition of any one of Embodiments [47] to [53] having a pH of 3.95±0.1.

Embodiment [55] relates to the composition of Embodiment [54] wherein the pH is 3.95±0.05.

The following non-limiting, illustrative examples provide detailed conditions and materials that can be useful in the practice of the present invention. Unless specifically indicated to the contrary, any preparation and testing was done at room temperature, i.e., an ambient temperature of from 20° to 25° C.

### Examples

Citric acid was selected as the acid in the composition in view of the previously described biocompatibility considerations. Trisodium citrate was selected as the conjugate base so as to provide maximum buffering capacity.

For skin/tissue contact applications, pH≈3 (approximately the pH of many sweet white wines) can irritate sensitive tissues, while pH≈4 (approximately the pH of some dry red wines) generally is considered non-irritating.

Based on the foregoing and the aforedescribed peritoneal cavity osmolarity limits, the base formulation used for initial testing employed 34.1 g anhydrous citric acid and 31.3 g trisodium citrate (this was the amount of citrate actually in the dihydrate form purchased from the supplier), diluted to 1 L with purified water. (Any water of hydration in the acid(s) or conjugate base(s) is irrelevant to efficacy because it merely becomes part of the solvent component when the solutes dissolve. Thus, the presence or absence of water of hydration in these types of components need only be accounted for when calculating moles and osmoles for purposes of pH and effective solute concentration.)

The initial base formulation had a calculated effective solute concentration of ˜600 mOsm/L. It was titrated with a strong acid to reach pH≈4.

One anionic and one cationic surfactant often used in oral care formulations, where consumption is a known and accepted risk of use, were selected: SLS and CPC.

Based on the foregoing, four compositions were prepared for testing against two ubiquitous bacteria of interest, specifically, S. aureus and P. aeruginosa. The solute components of these compositions are summarized in the following table, with each numerical value representing grams per liter. Water was the only solvent component.

TABLE 2
Initial compositions, solutes

| | | Conjugate | Anionic | Cationic |
|---|---|---|---|---|
| | Acid | base | surfactant | surfactant |
| 1 | 34.1 | 31.3 | 0.5 | — |
| 2 | 34.1 | 31.3 | 1.0 | — |
| 3 | 34.1 | 31.3 | — | 0.12 |
| 4 | 34.1 | 31.3 | — | 0.25 |

These formulations were evaluated in a drip flow biofilm reactor model (3-hour treatment time), using 72-hour biofilms of each of the two aforementioned bacteria. Results are shown below, where Control represents a normal saline solution.

TABLE 3
Drip flow model results

Log reduction in colony forming units

| | S. Aureus | P. aeruginosa |
|---|---|---|
| Control | 0.5 | −0.1 |
| 1 | 5.1 | 3.2 |
| 2 | 6.7 | 3.8 |
| 3 | 3.2 | 1.2 |
| 4 | 3.2 | 1.8 |

Based on the foregoing, a larger amount of a composition based on Example 2 was prepared from 32.5 g anhydrous citric acid, 35.7 g trisodium citrate dihydrate, 1.0 g SLS and 963.8 g water. Tabulated below are the amounts (in g) of each component of this composition, which had a pH of ˜4.0, a calculated effective solute concentration of 600 mOsm/L and a measured effective solute concentration (via freezing point depression) of 525 to 530 mOsm/L. This version used a slightly larger amount of citric acid so as to reduce the amount of titration with strong acid needed to reach the target pH. (As mentioned previously, the presence or absence of water of hydration for any of the solute components is not a limiting feature because the associated water merely becomes part of the solvent component, requiring only being taken into account for purposes of calculating effective solute concentration.

This composition (designated Example 5) was compared against prepared compositions targeted at duplicating the active ingredients of three commercially available aqueous products presently used to wash surgical wounds prior to approximation, each of which includes an indication about being rinsed or lavaged (typically with a saline solution) after introduction.
Comparative A—99.574% water, 0.4% NaCl, 0.025% HClO and 0.001% NaClO3, with all amounts w/v
Comparative B—0.05 g/L chlorhexidine gluconate
Comparative C—17.5 mL 10% (w/v) povidone iodine in 500 mL normal saline
   (Comparative C is a solution made and used by some surgeons.)

Efficacy testing was performed similarly to that described above in connection with obtention of the data tabulated in Table 3 but on a wider spectrum of bacteria. Comparative A was allowed to act on the bacterial biofilms for 300 seconds, while Comparatives B and C were allowed to act for 120 seconds; the dwell times for Comparatives A and B were taken from their respective labels. Each efficacy testing was done in triplicate.

Because the composition of Example 5 need not be washed or rinsed away prior to surgical wound approximation, its efficacy was evaluated using a variety of treatment (dwell) times. The tabulated data below uses the following abbreviations to represent approximate dwell times: 5a—120 seconds, 5b—900 seconds, 5c—1800 seconds, 5d—3600 seconds, 5e—7200 seconds, and 5f—10,800 seconds. Its data is presented in an x/y format, with x representing the mean logarithmic reduction relative to a control (a normal saline solution) and y representing standard deviation. The designations in the first column represent different types of bacteria, specifically,

TABLE 4
Log reductions in Colony Forming Units

| | Comp. A | Comp. B | Comp. C | 5a | 5b | 5c | 5d | 5e | 5f |
|---|---|---|---|---|---|---|---|---|---|
| M1 | 0.12/0.17 | 0.22/0.20 | 0.65/0.66 | 1.01/0.37 | 2.81/0.43 | — | — | — | 4.41/0.49 |
| M2 | 0.74/0.21 | 0.05/0.22 | 0.59/0.66 | 0.48/0.32 | 0.98/0.33 | — | — | — | 2.05/0.63 |
| M3 | — | 0.22/0.17 | 0.65/0.33 | 0.63/0.19 | 0.87/0.40 | 1.63/1.02 | 2.19/0.77 | 2.74/2.14 | 4.06/0.83 |
| M4 | — | 0.82/0.52 | 1.37/0.22 | 0.46/0.11 | 0.02/0.14 | 0.37/0.51 | 0.59/0.10 | 1.18/0.40 | 1.71/0.29 |
| M5 | — | 0.76/0.41 | 0.64/0.23 | 0.19/0.15 | 0.46/0.48 | 0.77/0.43 | 0.94/0.79 | 2.29/0.07 | 3.05/1.86 |

M1: S. aureus (ATCC 10943, MRSA)
M2: P. aeruginosa (ATCC 215)
M3: S. epidermidis (ATCC 35984)
M4: E. coli (ATCC 25922)
M5: C. acnes (ATCC 6919)

The data of Table 4 show the benefit of a composition which need not be washed out prior to wound approximation. While the composition of Example 5 had efficacy of a similar order of magnitude to the comparatives when used for dwell times similar to those permitted with those compositions, the fact that a composition of the type of Example 5 need not be rinsed or lavaged out of the wound cavity prior to approximation—and in fact can remain in the wound cavity even after wound approximation—resulted in a tremendous efficacy advantage. Additionally, because the composition was pH buffered and had a relatively high effective solute concentration, it continued to act over whatever extended dwell time was permitted. This effect is most apparent with reference to the 5f column, where the composition had at least a 2 log reduction versus control in CFUs for each of the five types of bacterial biofilms tested.

A composition was tested for safety in a mammalian model (Oryctolagus cuniculus albino rabbits, New Zealand White strain). The full report was included as an appendix to the specification of the priority application, U.S. patent appl. No. 63/091,421, but one result of the report is briefly summarized here.

The ingredients of the tested composition (pH=4.0, calculated osmolarity=670 mOsm/L) were as follows: 32.5 g anhydrous citric acid, 35.7 g trisodium citrate dihydrate, 1.0 g SLS, and 962.0 g water. The safety of this composition was compared to that from a negative control composition (hypertonic saline, 3%) to evaluate acute local irritation potential. Each composition was applied in 20 mL aliquots to rabbit tissue in situ for 10-11 minutes. This time simulated a worst-case scenario exceeding the intended clinical exposure of application followed by immediate aspiration.

The tissues evaluated were articular cartilage, cranial dura mater, mesentery, and pericardium. The animals underwent surgery to expose the required tissue for exposure to either the tested composition or the control composition. After the ˜10-minute exposure, the tested composition or the control composition was removed by blotting with sterile gauze, without rinsing prior to surgical approximation.

The tissues were evaluated histologically approximately 30 minutes, 24 hours, and 7 days after exposure to either the tested composition or the control composition. When compared to the control composition, the tested composition was found to be a non-irritant at these time points, for all tissue types tested.

These results provide evidence that the tested composition is non-irritating when used during surgery, even without rinsing or suctioning. That is, the tested composition was found to be non-irritating even when some of it remained in the surgical wound cavity during and after surgical wound approximation.

1: A method for treating a wound cavity in a mammalian subject, said method comprising:
a) providing a sterile, acidic liquid composition, said composition consisting of solvent and solute components and having an effective solute concentration of from 0.3 to 0.7 Osm/L and a pH of from 3.7 to 4.2;
b) prior to approximation of said wound, introducing said composition to said wound cavity; and
c) permitting at least a portion of said composition to reduce bioburden in said wound cavity during and after approximation of said wound.

2: The method of claim 1 wherein said composition has an effective solute concentration of from 350 to 590 mOsm/L.

3: The method of claim 1 wherein said composition has an effective solute concentration of from 450 to 680 mOsm/L.

4: The method of claim 1 wherein said composition has a pH of from 3.85 to 4.05.

5: The method of claim 1 wherein said solvent component consists of purified water.

6: The method of claim 1 wherein all solutes in said solute component are pharmaceutical grade.

7: The method of claim 4 wherein said solute component comprises or consists of a buffer system and an ionic surfactant.

8: The method of claim 7 wherein said buffer system comprises dissociation products of a carboxylic acid and a conjugate base of a carboxylic acid.

9: The method of claim 8 wherein said buffer system consists of dissociation products of at least one carboxylic acid and at least one conjugate base of at least one carboxylic acid.

10: The method of claim 9 wherein said at least one carboxylic acid is citric acid and wherein said at least one conjugate base is a citrate.

11: The method of claim 10 wherein said buffer system comprises dissociation products of from 25 to 40 g/L citric acid and from 30 to 45 g/L of a citrate that comprises three alkali metal ions.

12: The method of claim 10 wherein said composition comprises from 0.7 to 1.9 g/L ionic surfactant.

13: The method of claim 12 wherein said composition is provided in a container that comprises at least one access point.

14: The method of claim 12 wherein said ionic surfactant is an anionic surfactant.

15: The method of claim 14 wherein said anionic surfactant is sodium lauryl sulfate.

16: The method of claim 1 wherein said composition is introduced by an emergency medical service provider, wherein at least some of the bioburden reduction occurs prior to or during transport of said mammalian subject.

17: The method of claim 1 wherein said composition is introduced during an operation in a surgical theater, wherein the bioburden reduction occurs before, during and after said wound approximation.

18-33. (canceled)
